(19) United States
(12) Patent Application Publication
(10) Pub. No.: US 2002/0099403 A1
(43) Pub. Date: Jul. 25, 2002

(54) T-pellet for acupuncture

(76) Inventor: Tae Woo Yoo, Seoul (KR)

Correspondence Address: Richard M. Goldberg, Suite 419, 25 East Salem Street, Hackensack, NJ, 07601, US

(21) Appl. No.: 09/838,492

(22) Filed: Apr. 19, 2001

(30) Foreign Application Priority Data
Jan. 20, 2001 (KR) 2001-1689

(51) Int. Cl. A61B 17/34

(52) U.S. Cl. 606/189

(57) ABSTRACT

A T-pellet for acupuncture comprising: an adhesive tape with an adhesive surface on one side; a ring of a T-pellet to be attached on the adhesive surface of said adhesive tape; and a gold plate with a small hole in the center to be inserted by the end portion of said T-pellet.

### BACKGROUND OF THE INVENTION

1. Field of the Invention

The present invention relates to a T-pellet for acupuncture, and more particularly, to a T-pellet for acupuncture for providing an enhanced pressure stimulation effect by the T-pellet, a scattering effect of excessive body current and promoting a fluent body current circulation effect.

A human heart creates a body current of 10 micro ampere and transmits into the whole body. If the body current is transmitted into all the organs in a good balance, the body feels good and has no disease. But in case that excessive currents are transmitted into some parts, the body might get fever, pain, inflammation and so on. Conversely, in case that some parts get short of required currents, the body might experience psychroalgia, malfunction, paralysis, blood circulation disorder, trembling, spasm and so on.

2. Description of the Prior Art

In general, electronic needles were used in acupuncture therapy in order to control the imbalance of body current as above. The electronic needles were helpful to control the imbalance of body currents, but sometimes cause dizziness, fatigue, fever, paralysis, hallucination, powerlessness, since these electronic needles create pretty strong currents of between minimum 100 micro ampere and maximum 800 micro ampere.

An alternative to the electronic needles was a gold string inserted into Meridian points in the skin or the affected parts so as to keep balance in transmitting body currents. This treatment could be also dangerous to the body due to lack of safety and was not economical since the string is made of expensive gold.

And another solution to the above problem was a finger pressure device which is the prior art of the present invention, including an adhesive tape with a small hole in the center to be inserted by an end portion of a T-pellet. But the T-pellet of this device provides just pressure-stimulation effects to the Meridian points of the skin or the affected parts, but does not offer any other treatment effects.

### SUMMARY OF THE INVENTION

The present device was designed to overcome the disadvantages described as above. An object of the present invention is to provide a T-pellet for acupuncture which has enhanced pressure stimulation effects due to the end portion of T-pellet.

Another object of the present invention is to provide a T-pellet for acupuncture which has a scattering effect of excessive body current in some parts of the body, since the gold plate of the T-pellet for acupuncture has a great electric conduction. In case that excessive currents are transmitted into some parts, the body might get fever, pain, inflammation and so on.

The third object of the present invention is to provide a T-pellet for acupuncture which promotes a fluent body current conduction in some parts of the body. In case that a person gets short of required body current, the body might experience psychroalgia, malfunction, paralysis, blood circulation disorder, trembling, spasm and so on.

### BRIEF DESCRIPTION OF THE DRAWINGS

The other objects and features of the present invention will be hereinafter explained in detail with reference to the accompanying drawings, wherein:

FIG. 1 is a disassembled perspective view of the present invention.

FIG. 2 is a perspective view of the present invention.

FIG. 3 is a cross-sectional view showing a state of a use of the present invention.

FIG. 4 is a disassembled perspective view of another embodiment of the present invention.

FIG. 5 is a perspective view of FIG. 4.

FIG. 6 is a cross-sectional view of FIG. 4.

FIG. 7 is a disassembled perspective view of another embodiment of the present invention.

FIG. 8 is a perspective view of FIG. 7.

FIG. 9 is a cross-sectional view of FIG. 7.

FIG. 10 is a disassembled perspective view of another embodiment of the present invention.

FIG. 11 is a perspective view of FIG. 10. FIG. 12 is a cross-sectional view of FIG. 10.

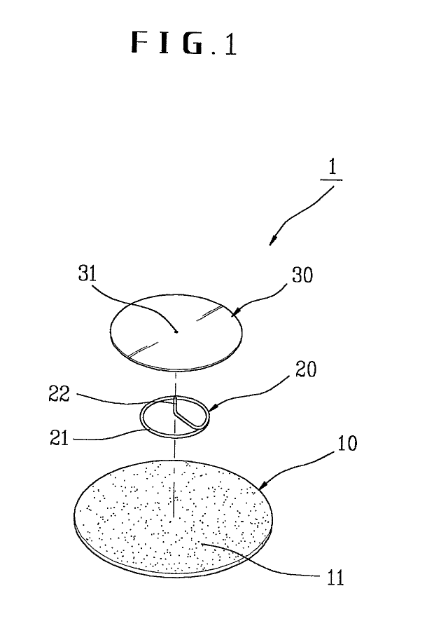

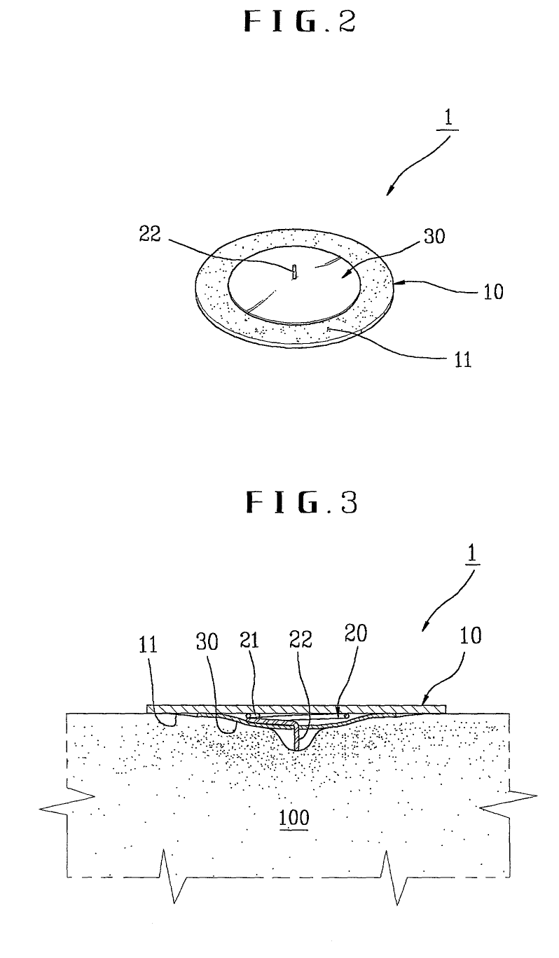

### DESCRIPTION OF THE PREFERRED EMBODIMENTS

A T-pellet 1 for acupuncture according to the present invention includes an adhesive tape 10 with an adhesive surface 11 on one side; a T-pellet 20 with a ring 21 and an end portion 22 to be attached on the adhesive surface 11 ; and a gold plate 30 with a small hole 31 in the center to be inserted by the end portion 22 of the T-pellet 20.

The numeral 100 refers to the skin.

The ring 21 of the T-pellet 20 is attached on the adhesive surface 11 of the adhesive tape 10, and the gold plate 30 is attached on the ring 21 of the T-pellet 20. The end portion 22 of the T-pellet 20 is inserted through the small hole 31 of the gold plate 30. Only the end portion 22 of the T-pellet 20 is extended outwardly from the small hole 31 of the gold plate 30.

As shown in FIG. 3, the T-pellet 1 is attached at the Meridian points or the affected parts of the skin 100. The end portion 22 of the T-pellet 1 is placed at the Meridian points and the gold plate 30 is also placed directly on the skin. The end portion 22 placed at the Meridian points provides pressure-stimulation effects to the Meridian points and gold of the gold plate 30 has a power of controlling body currents due to its electric transmission.

In traditional chinese medicine, gold is used for medicine pills. For example, invigorating pills are wrapped with thin gold foils and users take the pills without peeling the gold foils. And gold powder is also swallowed by patients because gold is believed to do good for the five viscera and the six entrails and have an effect of stabilizing human spirits.

In case that excessive body currents are transmitted into some parts, the body might get fever, pain, inflammation and so on. The gold plate 30 of the T-pellet 1 has an effect of scattering excessive currents. Conversely, in case that some parts of the body get short of required currents, the body might experience psychroalgia, malfunction, paralysis, blood circulation disorder, trembling, spasm and so on. The gold plate 30 of the T-pellet 1 has an effect of promoting transmission of body currents.

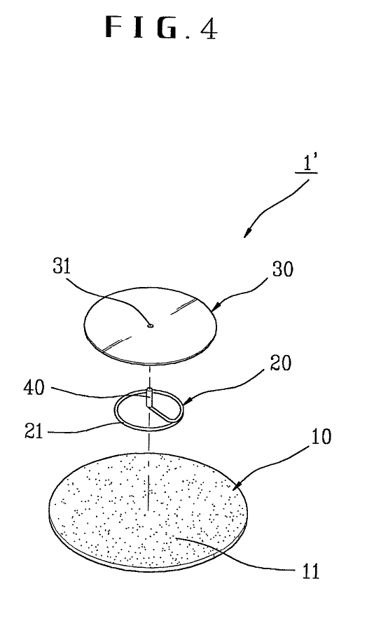

FIG. 4 is another embodiment of the present invention, showing a T-pellet 1′ for acupuncture consisting of an adhesive tape 10 with an adhesive surface 11 on which a ring 21 of a T-pellet 20 is attached. The T-pellet 20 has a gold coating portion 40 formed at the end of the ring 21 and inserted through the small hole 31 of the gold plate 30 which is attached on the ring 21.

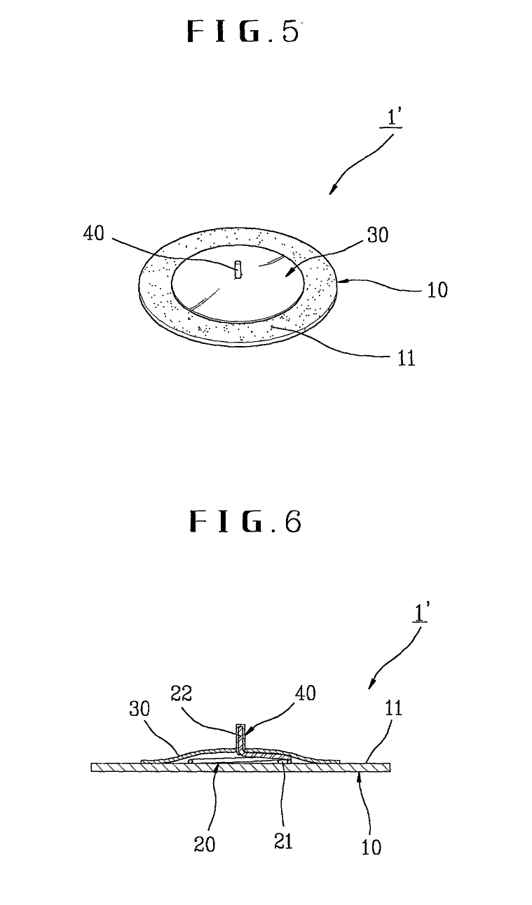

As shown in FIG. 5, the T-pellet 1′ is attached at the Meridian points or the affected parts of the skin 100 by the adhesive tape 10, and the gold coating portion 40 at the end of the T-pellet 20 and the gold plate 30 provide pressure-stimulation effects on the skin and control body currents at the same time.

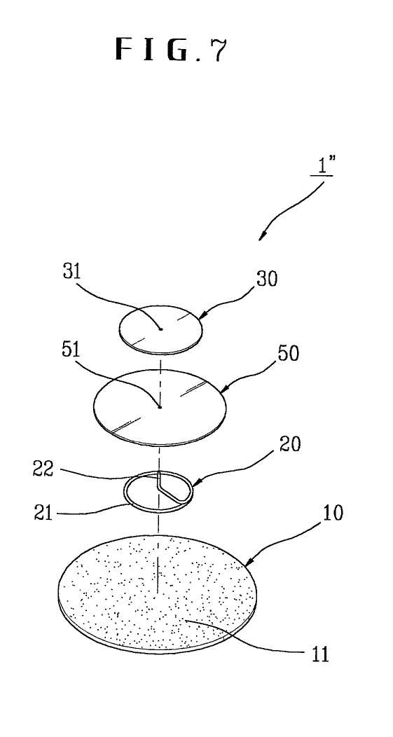

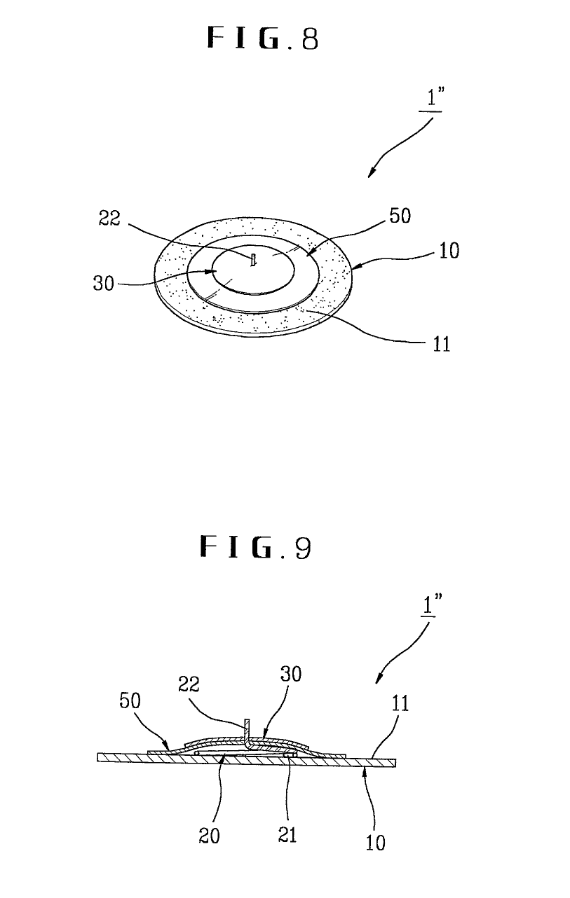

FIG. 7 is another embodiment of the present invention, showing a T-pellet 1″ for acupuncture consisting of an adhesive tape 10 with an adhesive surface 11 on which a ring 21 of a T-pellet 20 is placed. A silver plate 50 with a small hole 51 in the center is attached on the adhesive tape 10 with the small hole 51 of the silver plate 50 inserted by the end portion 22 of the T-pellet 20. A gold plate 30 with a small hole 31 in the center is placed on the silver plate 50. The small hole 31 of the gold plate 30 is also inserted by the end portion 22 of the T-pellet 20.

As shown in FIG. 9, the T-pellet 1″ is placed at the Meridian points or the affected parts of the skin 100. The end portion 22 placed at the Meridian points or the affected parts offers a pressure-stimulation effect to the skin 100. The silver plate 50 has an ionization tendency due to its material of silver and keeps the affected areas warm. The gold plate 30 is able to control body currents due to its material of gold.

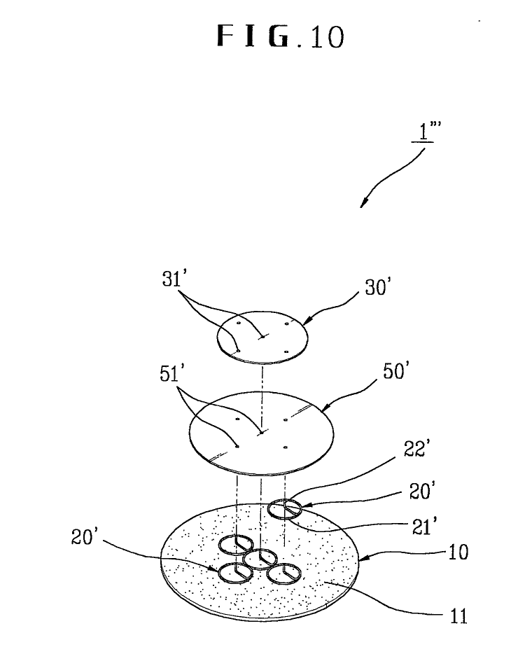

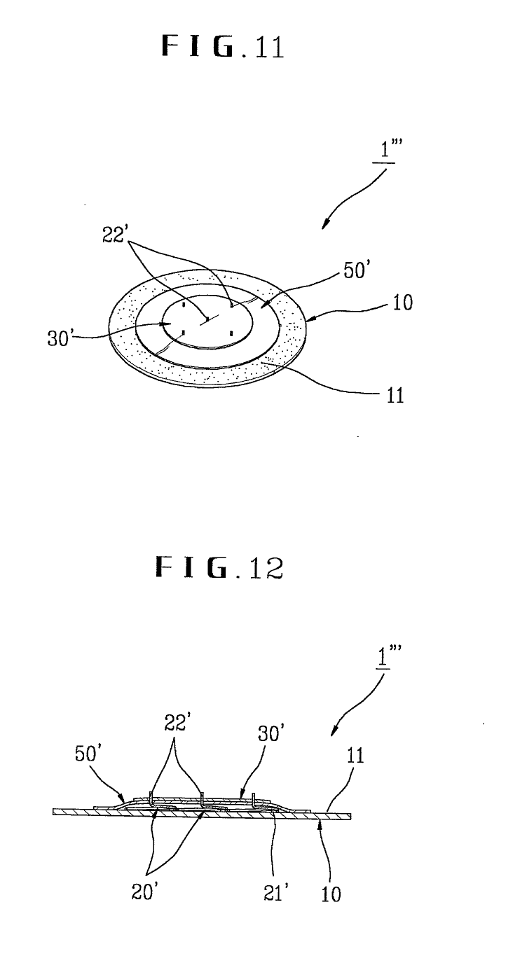

FIG. 10 is another embodiment of the present invention, showing a T-pellet 1″ for acupuncture consisting of an adhesive tape 10 with an adhesive surface 11 on one side, a plurality of T-pellets 20′ to be attached on the adhesive surface 11 of the adhesive tape 10, a silver plate 50′ with a plurality of small holes 51′ in the centers to be inserted by the end portions 22′ of the T-pellets 20′, respectively, and a gold plate 30′ with a plurality of small holes 31′ to be inserted by the end portions 22′ of the T-pellets 20′, respectively.

As shown in FIG. 12, the T-pellet 1′″ is placed at the Meridian points or the affected parts of the skin 100. The end portions 22′ offer a pressure-stimulation effect to the skin 100. The silver plate 50′ has an ionization tendency due to its material of silver and keeps the affected areas warm. The gold plate 30′ is able to control body currents due to its material of gold.

What is claimed is:

1. A T-pellet for acupuncture comprising:
an adhesive tape with an adhesive surface on one side;
a ring of a T-pellet to be attached on the adhesive surface of said adhesive tape; and
a gold plate with a small hole in the center to be inserted by the end portion of said T-pellet.

2. A T-pellet for acupuncture according to claim 1, wherein a gold coating portion is formed on said end portion of said T-pellet attached on the adhesive surface of said adhesive tape, and inserted through the small hole in the center of said gold plate attached on the adhesive surface of said adhesive tape.

3. A T-pellet for acupuncture according to claim 1, comprising,
an adhesive tape with an adhesive surface on one side;
a ring of a T-pellet to be attached on the adhesive surface of said adhesive tape;
a silver plate with a small hole in the center to be inserted by the end portion of said T-pellet; and
a gold plate with a small hole in the center to be inserted by the end portion of said T-pellet.

4. A T-pellet for acupuncture according to claim 1, comprising,
an adhesive tape with an adhesive surface on one side;
a plurality of rings of a plurality of T-pellets to be attached on the adhesive surface of said adhesive tape;
a silver plate with a plurality of small holes in the centers to be inserted by the end portions of said T-pellets, respectively; and
a gold plate with a plurality of small holes in the centers to be inserted by the end portions of said T-pellets, respectively.
